SEQUENCE LISTING (6 sequences)

SEQ ID NO: 1 - protein, 939 aa, Homo sapiens
MDDSEVESTASILASVKEQEAQFEKLTRALEEERRHVSAQLERVRVSPQDANPLMANGTLTRRHQNGRFVGDADLERQKFSDLKLNGPQDHSHLLYSTIPRMQEPGQIVETYTEEDPEGAMSVVSVETSDDGTTRRTETTVKKVVKTVTTRTVQPVAMGPDGLPVDASSVSNNYIQTLGRDFRKNGNGGPGPYVGQAGTATLPRNFHYPPDGYSRHYEDGYPGGSDNYGSLSRVTRIEERYRPSMEGYRAPSRQDVYGPQPQVRVGGSSVDLHRFHPEPYGLEDDQRSMGYDDLDYGMMSDYGTARRTGTPSDPRRRLRSYEDMIGEEVPSDQYYWAPLAQHERGSLASLDSLRKGGPPPPNWRQPELPEVIAMLGFRLDAVKSNAAAYLQHLCYRNDKVKTDVRKLKGIPVLVGLLDHPKKEVHLGACGALKNISFGRDQDNKIAIKNCDGVPALVRLLRKARDMDLTEVITGTLWNLSSHDSIKMEIVDHALHALTDEVIIPHSGWEREPNEDCKPRHIEWESVLTNTAGCLRNVSSERSEARRKLRECDGLVDALIFIVQAEIGQKDSDSKLVENCVCLLRNLSYQVHREIPQAERYQEAAPNVANNTGPHAASCFGAKKGKDEWFSRGKKPIEDPANDTVDFPKRTSPARGYELLFQPEVVRIYISLLKESKTPAILEASAGAIQNLCAGRWTYGRYIRSALRQEKALSAIADLLTNEHERVVKAASGALRNLAVDARNKELIGKHAIPNLVKNLPGGQQNSSWNFSEDTVISILNTINEVIAENLEAAKKLRETQGIEKLVLINKSGNRSEKEVRAAALVLQTIWGYKELRKPLEKEGWKKSDFQVNLNNASRSQSSHSYDDSTLPLIDRNQKSDKKPDREEIQMSNMGSNTKSLDNNYSTPNERGDHNRTLDRSGDLGDMEPLKGTTPLMQKI

SEQ ID NO: 2 - DNA, 6226 bp, Homo sapiens
  CDS (537)..(3356)
ctgccagatcagtttgtcaccacccaggctcccttgcctttggctgggtgcaacttccattttaggtgttggatctgagggggaaaaaaaagagagagggagagagagagaaagaagagcaggaaagatcccgaaaggaggaagaggtggcgaaaaatcaactgccctgctggatttgtctttctcagcaccttggcgaagccttgggtttctttcttaaaggactgatttttagaactccacatttgaggtgtgtggcttttgaagaaaatgtatgtactgacgggaaaaggaagataagcaagtcgaatttttgtcttacgctctctccttcctgcttcctccttgctgtggtggctgggatgctccttccatgattttttgaatctagactgggctgttctctgtgttaaaccaatcagttgcgaccttctcttaacagtgtgaagtgagggggtctctctccctccttctccttcctctgtgattcaccttcctttttaccctgccctgcggcggctccgccccttaccttcatggacgactcagaggtggagtcgaccgccagcatcttggcctctgtgaaggaacaagaggcccagtttgagaagctgacccgggcgctggaggaggaacggcgccacgtctcggcgcagctggaacgcgtccgggtctcaccacaagatgccaacccactcatggccaacggcacactcacccgccggcatcagaacggccggtttgtgggcgatgctgaccttgaaagacagaaattttcagatttgaaactcaacggaccccaggatcacagtcaccttctatatagcaccatccccaggatgcaggagccggggcagattgtggagacctacacggaggaggatcctgagggagccatgtctgtagtctctgtggagacctcagatgatgggaccactcggcgcacagagaccacggtcaagaaagtagtgaagactgtgacaacacggacagtacagccagtcgctatgggaccagacgggttgcctgtggatgcttcatcagtttctaacaactatatccagactttgggtcgtgatttccgcaagaatggcaatgggggacctggtccctatgtggggcaagctggcactgctacccttcctaggaacttccactaccctcctgatggttatagtcgccactatgaagatggttatccaggtggcagtgataactatggcagtctgtcccgggtgacccgcattgaggagcggtataggcccagcatggaaggctaccgggcacctagtagacaggatgtgtatgggccccaaccccaggttcgggtaggtgggagcagcgtggatctgcatcgctttcatccagagccttatgggctagaggatgaccagcgtagtatgggctatgatgacctggattatggtatgatgtctgattatggcactgcccgtcggactgggacaccctctgaccctcgtcggcgcctcaggagctatgaagacatgattggtgaggaggtgccatcggatcaatactactgggctcctttggcccagcatgagcgaggaagtttagcaagcttggatagcctgcgcaaaggagggcctccacctcctaattggagacagccagagctgccagaggtgatcgccatgcttggattccgcttggatgctgtcaagtccaatgcagctgcatacctgcaacacttatgctaccgcaatgacaaggtgaagactgacgtgcggaagctcaagggcatcccagtactggtgggattgttagaccatcccaaaaaggaagtgcaccttggagcctgtggagctctcaagaatatctcttttggacgtgaccaggataacaagattgccataaaaaactgtgatggtgtgcctgcccttgtgcgattgcttcgaaaggctcgtgatatggaccttactgaagttattaccggaaccctgtggaatctttcatcccatgactcaatcaaaatggagattgtggaccatgcactgcatgccttgacagatgaagtgatcattcctcattctggttgggagcgggaacctaatgaagactgtaagccacgccatattgagtgggaatcggtgctcaccaacacagctggctgccttaggaatgtaagctcagagaggagtgaagctcgccggaaacttcgggaatgtgatggtttagttgatgccctcattttcattgttcaggctgagattgggcagaaggattcagacagcaagcttgtagagaactgtgtttgccttcttcggaacttatcatatcaagttcaccgggagatcccacaggcagagcgttaccaagaggcagctcccaatgttgccaacaatactgggccacatgctgccagttgctttggggccaagaagggcaaagatgagtggttctccagagggaaaaaacctatagaggatccagcaaacgatacagtggatttccctaaaagaacgagtccagctcgaggctatgagctcttatttcagccagaggtggttcggatatacatctcacttcttaaggagagcaagactcctgccatcctagaagcctcagctggagctatccagaacttgtgtgctgggcgctggacgtatggtcgatacatccgctctgctctgcgtcaagagaaggctctttctgccatagctgacctcctgactaatgaacatgaacgggtggtgaaagctgcatctggagcactgagaaacctggctgtggatgctcgcaacaaagaattaattggtaaacatgctattcctaacttggtaaagaatctgccaggaggacagcagaactcctcttggaatttctctgaggacactgtcatctctattttgaacactatcaacgaggttatcgctgagaacttggaggctgccaaaaagcttcgagagacacagggtattgagaagctggtgttgatcaacaaatcagggaaccgctcagaaaaagaagttcgagcagcagcacttgtattacagacaatctggggatataaggaactgcggaagccactggaaaaagaaggatggaagaaatcagactttcaggtgaatctaaacaatgcttcccgaagccagagcagtcattcatatgatgatagtactctccctctcattgaccggaaccaaaaatcagataagaaacctgatcgggaagaaattcagatgagcaatatgggatcaaacacaaaatcactagataacaactattccacaccaaatgagagaggagaccacaatagaacactggatcgatcgggggatctaggcgacatggagccattgaagggaacaacacccttgatgcagaagatttagcaccactatctccgttccatctgggcttatatgtacttttattttttggtggtgaaattgactgatgattttcctttttcttcgctggactattgtgccaactgccaggctgcctcctgcccttacagccctaagtggctgccttctttccatcaactcccaacttcttcctgtgaagtttaattgtctcaacgcctccccctcccccattccctccatttttctcccaagaaacctgactcaattatttgcatattttgagaaactgctgcagattagttctttttgccagttttccctggaactcctggccttttgtggaggggagggatggagagaataggaatcttcactagaagccgtgggaagaattggaagttacatgctgtatatgcaatgtccagcagtctgataaactgacgattcttaatcaagatttttttcctgatggggaagggacttttattttcttttagagaggggaaagtgtgagctcttcccttattcctaatggctatttttgaagcaaagaaggccagcaacattggcacatgccacctggcaaaggacccttgagtaagtgaaggtctcctaaaactgggattaagaaaccttgctctcctcatctccaaggcagggaccatcaagaacctacagactccatctcttctgcaagcctcatgccaaccctgggctattgctgctgccccttaaacacaggctgtccttaacccacctctcctgccctgtgatatgtctgctgagttggcctggccatttccaagaggctgtagaaaggggagaatgtcaaggaagacttttggtagagaaggagcagaaagatgtgtttttgggaagaagaagacctctaggaggagctagtaggaatgtacatgaagcaattagtctgaaactggcttccccactcccccgtttctccttttcctatccttataggcctgtcccttgcctctgccctggattggttggcaaactataggacttgatgtacataactcctgtcccttttcccttacaaggtggggattgcccctggctttgcctcttctttgtgcctttggcctggggtgcatctcctcccgcccttccatgtgcctttctttgcctctgcagtctcatttctcataattttgcaaattatattttgttgctttcttacctactattggccctaaatagcagaaagaagagaagtgaccgagagaacctcagattcttcattgaggattggtatagccatgatttcagtcatagcaagcttttgctcaacagcatatgggtgggatttggcaaaaatcctattctgatgaatctcaaagtaaggctggtaagagaagtgagtggtgtgactcttactccttaggtgcccagaatttaccatcatctctgaaggagttacagggaagtggtctccccaattctcccctccctccagtattgccccctctcactttagcatatattaattagcaggttgggctagagaaatcagctgctatgcgggttgattattattattatttctaatccttttccttatttgccttctactccccttaatctaatctaaaagctctgttccatgcaactggagttccttatccctctcttccccttcccttatatattgaggctatggggtaggagaaaagtgcacaacccaccaccccctctactcgtgcattaaaatttcttatttacccttttcccccttcccatttcttcccactttcatctaccttttctggcaaaaaggagccttttgctctctgtgaccctaagagcacactgcacagggaaaattgccccatccagacctggctccactcttgatctctcttgtcctcttctgctcttttcctggtgctcttttttctcggtggggtgtgggtaatagaacagccgtgggcttttggggacctttaacttttttttctctcttttgtttataaaaaacactaaacattcaattccagagaacccaaaatcccaccttcccaccgaacactactaaggggcttgtgttctgctccataccttttctcttttctttctgtcttgttaatgcttttaaaaacaaatgagttttttatataaataaagtttttaaagtgtgtatgtggggggtctgtgtcatttcttcacttcaagctgttatttcttccctgctttgcatctttgttacttccttatgtatcagtgtcctttccagagcaaccagaaggaggttataccaggatttattttgagctcagccccaactctttatcaagcaacattcttgttaactatatgtgaaacattttttcttctgaagattcttaaaaattgaatgtggctgaagttgaacatgggagcttattgctaatttagagataggaaactgaagcataaagaattaatgacttactttaattactggaattcttctgcaacatttgacaaaactaaccttgaataaggcccactgtaatacgtagctctcttaaatataacacttaggactagaagattagaaactaccaatcccaactacgtaataggaaaatgtaggatcaaaaggcccatgtatataagtactgaccactgggccataatgttgcttctcaggctatatgcagtcctttagtcagaagtcaataggcctatttattaatattttacagaccatattacctggattaccagggactatctttgctgcagagatcaagggttaagatctatgggaagatacttatttttctgaggtccttatgtcctgtcatataattaaagactcaagagaatttatgtgaaatgctttctgtatgccccaatctttagattaaaattatatacctgctcct

SEQ ID NO: 3 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 4 - DNA, 2765 bp, Homo sapiens
  CDS (249)..(1652)
tgggacaggcagctccggggtccgcggtttcacatcggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataaccaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccggttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgcccccagatgcctcctctgtcctcattcttctctcccacacaagcagtctttttctacagccagtaaggcagctctgtcrtggtagcagatggtcccattattctagggtcttactctttgtatgatgaaaagaatgtgttatgaatcggtgctgtcagccctgctgtcagaccttcttccacagcaaatgagatgtatgcccaaagcggtagaattaaagaagagtaaaatggctgttgaagc

SEQ ID NO: 5 - protein, 448 aa, Homo sapiens
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTTFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 6 - DNA, 2236 bp, Homo sapiens
  CDS (368)..(1714)
cgagcggcggcggagcaggcatttccagcagtgaggagacagccagaagcaagctattggagctgaaggaacctgagacagaagctagtcccccctctgaattttactgatgaagaaactgaggccacagagctaaagtgacttttcccaaggtcgcccagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtgcttccagaggcagggctatgctcacattcatggcctctgacagcgaggaagaagtgtgtgatgagcggacgtccctaatgtcggccgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgacattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctgagggacatggtgtgccacaggctgcaagctgcagggaattttcattggatgcagttgtatagttttacactctagtgccatatatttttaagacttttctttccttaaaaaataaagtacgtgtttacttggtgaggaggaggcagaaccagctctttggtgccagctgtttcatcaccagactttggctcccgctttggggagcgcctcgcttcacggacaggaagcacagcaggtttatccagatgaactgagaaggtcagattagggcggggagaagagcatccggcatgagggctgagatgcgcaaagagtgtgctcgggagtggcccctggcacctgggtgctctggctggagaggaaaagccagttccctacgaggagtgttcccaatgctttgtccatgatgtccttgttattttattgcctttagaaactgagtcctgttcttgttacggcagtcacactgctgggaagtggcttaatagtaatatcaataaatagatgagtcctgttagaaaaa

(19) United States
(12) Patent Application Publication
(10) Pub. No.: US 2005/0101766 A1
(43) Pub. Date: May 12, 2005

(54) Method of identifying modulators of presenilin

(76) Inventors: Walter Blackstock, Hertfordshire (GB); Richard Stephen Hale, Prestwood (GB); Adele Rowley, St Albans (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/221,291

(22) PCT Filed: Mar. 9, 2001
(86) PCT No.: PCT/GB01/01059

(30) Foreign Application Priority Data
Mar. 10, 2000 (GB) 0005895.8

(51) Int. Cl. C07K 14/705

(52) U.S. Cl. 530/350

(57) ABSTRACT

A method of identifying a modulator of presenilin function, the method comprising: (i) providing (a) a polypeptide capable of binding a presenilin, which polypeptide comprises the amino acid sequence of SEQ ID NO: 1 or a functional variant thereof, or a fragment of either thereof which is capable of binding to presenilin; (b) a presenilin or a variant thereof or a fragment of either thereof capable of binding to a polypeptide which comprises the amino acid sequence of SEQ ID NO: 1; (c) a test substance under conditions that would permit binding of a polypeptide (a) to a presenilin (b) in the absence of the test substance; (ii) monitoring presenilin mediated activity; and (iii) determining thereby whether the test substance is a modulator of presenilin activity, a modulator identified by a method of the invention and use of a modulator identified by a method of the invention in the treatment of Alzheimer's disease.

### FIELD OF THE INVENTION

This invention relates to methods of identifying modulators of presenilin or catenin p120 or variants thereof and their use in the treatment of conditions in which abnormal activity of presenilin or catenin p120 is implicated such as Alzheimer's disease.

### BACKGROUND TO THE INVENTION

Presenilin 1 and the closely related presenilin 2 (PS1 and PS2) are membrane proteins predicted to span the membrane eight times. Presenilins are ubiquitously expressed at low levels and are located within the cell primarily in the endoplasmic reticulum (ER) and the Golgi apparatus, although presenilins have been localized to the plasma membrane in neuronal cells and COS-7 cells as well as to neuronal large dense-core granules and clathrin coated vesicles. Co-localization of PS-1 with kinetochores on the inner nuclear membrane has also been observed. Most cases of early onset familial Alzheimer's Disease (AD) are caused by mutations in the presenilin I gene. Presenilins play a role in the processing of amyloid precursor protein (APP) which is also implicated in early onset AD (reviewed by Selkoe, 1998). Co-immunoprecipitation experiments have shown that PS1 and PS2 interact directly with the immature forms of APP in the ER where the disease-associated amyloid β1-42 peptide (AP42) is probably generated (Xia et al., 1997; Weidemann et al., 1997). Links between presenilin function and the generation of Aβ42 via intra-membrane cleavage processing of APP have been clearly demonstrated in transgenic mouse models. Presenilins may play a role in the cleavage of APP through interaction with the as yet unidentified protease termed γ-secretase or, as has recently been suggested presenilins could be the γ-secretase themselves (reviewed in Haass and Mandelkov, 1999).

Presenilin is also involved in other biological pathways. One report suggests that PS1 directly binds tau and a tau kinase, glycogen synthase kinase 3beta (GSK-3beta) and proposes that the increased association of GSK-3beta with mutant PS1 leads to increased phosphorylation of tau (Takashima et al. 1998).

Presenilins are also known to play an important role in Notch signalling during early embryonic development and/or cellular differentiation (reviewed by Anderton, 1999). At least one recent report claims a direct interaction between Notch1 and PS1. These results might suggest that the genetic relationship between presenilins and the Notch signalling pathway derives from a direct physical association between these proteins in the secretory pathway (Ray et al., 1999).

Presenilins have also been demonstrated to interact with members of the armadillo protein family which are characterised by a series of 42 amino acid imperfect repeats that have been implicated in protein-protein interactions.

Yeast 2 hybrid experiments and/or immunoprecipitation experiments have previously shown that widely expressed β-catenins and plakoglobin/γ-catenins and another armadillo-protein, p0071 interact with PS1 (e.g. Kang et al., 1999; Stahl et al., 1999; Zhang et al., 1998; Zhou et al., 1997b; Yu et al., 1998). A neuronal-specific armadillo protein-neural plakophilin-related armadillo protein (NPRAP, a longer version of a sequence previously termed δ-catenin [Zhou et al., 1997a]) also interacts with PS1 (Levesque et al (1999)). Not all of these interactions are supported by all studies, for example Levesque et al (1999) did not find that plakoglobin/γ-catenin interacts with presenilin.

It has also been reported that loop regions of PS1 and PS2 interact with nonmuscle filamin (actin-binding protein 280, ABP280) and a structurally related protein (filamin homolog 1, Fh1) (Zhang et al., 1998).

Catenins were discovered as proteins that are linked to the cytoplasmic domain of transmembrane cadherins. Among these junctional plaque proteins are several members of the Armadillo gene family: β-catenin, plakoglobin, and catenin p120 (p120ctn). The human p120ctn gene comprises 21 exons, potentially encoding up to 32 protein isoforms as products of alternative splicing (Keirsebilck et al, 1998). Human isoforms, designated 1 to 4, differ from each other by the start codon used. Additional isoforms are derived from combinations with alternatively used exons A (exon 18) and B (20), near the end of the open reading frame, and also with exon C (11) in the middle of the open reading frame. Hence, the longest isoform is of type 1ABC and comprises 968 amino acid residues. The functional consequence of the observed multitude of p120ctn splice variants is unclear but there are tissue-specific expression level differences. The exon organization, which is not simply related to the Armadillo repeat structure, is very well conserved between the p120ctn gene and the related ARVCF gene, but not at all between these two genes and the P-catenin or plakoglobin genes.

### SUMMARY OF THE INVENTION

The present invention is based on a novel direct action between a presenilin and a protein, catenin p120.

The DNA and predicted amino acid sequence of catenin p120 have been previously deposited in public domain databases but this gene product has not previously been shown to interact with presenilin.

The interaction between presenilin and catenin p120 provides a new therapeutic intervention point in disorders involving defective presenilin function, and more specifically in Alzheimer's disease. In addition catenin p120 is now proposed as a target for identifying agents which may be useful in the treatment of Alzheimer's disease.

Accordingly the invention provides:
a method of identifying a modulator of presenilin function, the method comprising:
   (i) providing
      (a) a polypeptide capable of binding a presenilin, which polypeptide comprises the amino acid sequence of SEQ ID NO: 1 or a functional variant thereof or a functional fragment of either thereof which is capable of binding to a presenilin;
      (b) a presenilin or a variant thereof or a fragment of either thereof capable of binding to a polypeptide which comprises the amino acid sequence of SEQ ID NO: 1; and
      (c) a test substance
   under conditions that would permit binding of (a) to (b) in the absence of (c);
   (ii) monitoring presenilin mediated activity; and
   (iii) determining thereby whether the test substance is a modulator of presenilin activity.

In a further aspect, the invention provides a method for identification of a compound that modulates catenin p120 activity, which method comprises:
(i) contacting a catenin p120 polypeptide comprising
   (a) the amino acid sequence of SEQ ID NO: 1; or
   (b) a variant thereof or a fragment of either thereof which maintains a catenin p120 function; with a test compound and
(ii) monitoring for catenin p120 activity thereby determining whether the test compound is a modulator of catenin p120.

The invention also provides:
a modulator identified by a method according to any one of the preceding claims for use in a method of treatment of the human or animal body by therapy;
use of a modulator identified by a method according to any one of claims 1 to 10 in the manufacture of a medicament for the treatment or prophylaxis of a disorder that is responsive to modulation of presenilin activity;
use of a polypeptide capable of binding a presenilin, which polypeptide comprises the amino acid sequence of SEQ ID NO: 1 or a variant or fragment of SEQ ID NO: 1 which is capable of binding a presenilin in the manufacture of a medicament for the treatment, prophylaxis or diagnosis of a disorder that is responsive to modulation of presenilin activity;
use of a polynucleotide which encodes a polypeptide as defined in claim 13 or a catenin p120 polypeptide as defined in claim 9 comprising:
   (a) the sequence of SEQ ID NO: 2; or
   (b) a sequence that hybridizes to the complement of SEQ ID NO: 2; or
   (c) a sequence that is degenerate as a result of the genetic code with respect to a sequence defined in (a) or (b); or
   (d) a sequence that is complementary to a polynucleotide defined in (a), (b) or (c);
      in the manufacture of a medicament for the treatment, prophylaxis or diagnosis of a disorder that is responsive to modulation of presenilin activity.

Preferably the disorder responsive to presenilin modulation is Alzheimer's disease.

### BRIEF DESCRIPTION OF THE SEQUENCES

SEQ ID NO: 1 is the amino acid sequence of catenin p120.

SEQ ID NO: 2 comprises the amino acid sequence and encoding polynucleotide of catenin p120.

SEQ ID NO: 3 is the amino acid sequence of presenilin 1.

SEQ ID NO: 4 is the amino acid and encoding polynucleotide sequence of presenilin 1.

SEQ ID NO: 5 is the amino acid sequence of presenilin 2.

SEQ ID NO: 6 is the amino acid and encoding polynucleotide sequence of presenilin 2.

### DETAILED DESCRIPTION OF THE INVENTION

The invention provides a method for identifying a modulator of presenilin activity or a modulator of catenin p120 activity. A modulator may modulate the interaction between a presenilin and a catenin p120 isoform.

A presenilin for use in accordance with the invention herein referred to as presenilin (b) may comprise a naturally occuring presenilin such as PS-1 or PS-2 having the amino acid sequence of SEQ ID No: 3 or SEQ ID NO: 5 or may comprise a variant or fragment of such naturally occuring presenilin such as another unidentified isoform or splice variant which is homologous to or retains the desired function of a known presenilin. Such a variant or fragment of presenilin for use in the invention is one which is capable of binding to catenin p 120 having the sequence of SEQ ID NO: 1.

A polypeptide for use in accordance with the invention is one which capable of binding presenilin. The polypeptide, herein referred to as polypeptide (a) a catenin p120 isoform having the sequence of SEQ ID NO: 1 or a functional variant or a functional fragment thereof. A variant may comprise a naturally occuring isoform or splice variant. A variant or fragment of SEQ ID NO: 1 for use in accordance with the invention is capable of binding to presenilin and in particular presenilin 1.

To determine whether a variant or fragment of SEQ ID NO: 1 is capable of binding to a presenilin the variant or fragment can be contacted with a presenilin under conditions suitable for the formation of a complex between catenin p120 and presenilin. Similarly, to determine whether a presenilin, or variant or fragment thereof is capable of binding to catenin p120, the presenilin or fragment thereof can be contacted with catenin p120 under conditions suitable for the formation of a complex between catenin p120 and presenilin. Any one of the assays described herein can be carried out in the absence of a test substance to determine the binding capabilities of these proteins.

Proteins with naturally occurring amino acid sequences are preferred for use in the assays. Preferred proteins are human proteins but homologues from other mammalian species, or other animal species may be used. Any allelic variant or species homologue of the defined proteins may be used. References to a variant of the protein as described below relates to a variant of a presenilin or catenin p120. For all the proteins described herein for use in an assay of the invention, the ability of the variant to bind catenin p120 or presenilin as appropriate is preferably maintained.

Allelic variants and species homologues can be obtained by following the procedures described herein for the identification and production of proteins that bind catenin p120 or a presenilin as appropriate. It is also possible to use a nucleic acid probe as described herein to probe libraries made from human or other animal cells in order to obtain clones encoding allelic or species variants. The clones can be manipulated by conventional techniques to generate a polypeptide which can then be produced by recombinant or synthetic techniques known in the art.

Polypeptides that have been artificially mutated but retain catenin p120 or presenilin binding activity or other catenin p 120 or presenilin activity may also be used in the invention. Such mutants may be generated by techniques well known in the art, including site directed mutagenesis, random mutagenesis and restriction enzyme digestion and ligation. A protein for use in the invention preferably has at least 60% sequence identity to a natural protein, more preferably at least 70%, at least 80%, at least 90%, at least 95%, at least 97% or at least 99% sequence identity thereto over a region of at least 20, preferably at least 30, for instance at least 40, at least 60, at least 100 contiguous amino acids or over the full length of a natural protein. Amino acid substitutions may be made, for example from 1, 2 or 3 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |

| | |
|---|---|
| AROMATIC | H F W Y |

The entire protein sequence of each of the proteins used in the assay may be present. Fragments of the proteins and variants described above that retain the ability to bind to the second component in the binding assay, i.e. presenilin for catenin p120 and catenin p120 for presenilin may also be used in the invention. Alternatively variants or fragments of catenin p 120 which retain a function of catenin p120 may be used in assays to identify modulators of catenin p120. Preferred fragments will be at least 30, e.g. at least 100, at least 200, at least 300, at least 400, at least 500 or at least 600 amino acids in size. Preferred presenilin fragments are stable N- and C-terminal fragments that are generated in vivo by endoproteolysis (Capell et al., 1998; Yu et al., 1998). The term “isoform” is used herein to describe such variants and fragments of catenin p120 or presenilin.

The polypeptides for use in the invention may be chemically modified, e.g. post-translationally modified. For example, they may be glycosylated or comprise modified amino acid residues. They may also be modified by the addition of a signal sequence to promote their secretion from a cell where the polypeptide does not naturally contain such a sequence. The polypeptides may be tagged to aid detection or purification, for example using a HA, his8, his6, T7, myc or flag tag. Alternatively the polypeptides may be fusion proteins to aid purification or detection, for example, GST-fusion proteins may be used to aid purification from bacteria and GFP-fusion proteins may be used to aid detection. The polypeptide (a) and presenilin (b) may be tagged with different labels which may assist in identification of a polypeptide/presenilin complex.

Polypeptides for use in the invention may be in a substantially isolated form. It will be understood that the polypeptide may be mixed with carriers or diluents which will not interfere with the intended purpose of the polypeptide and still be regarded as substantially isolated. A polypeptide for use in the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention.

Polynucleotides

The invention provides a polynucleotide which encodes a polypeptide capable of binding a presenilin which polynucleotide consists essentially of:
(a) the sequence of SEQ ID NO: 2; or
(b) a sequence that hybridizes to the complement of SEQ ID NO: 2; or
(c) a sequence that is degenerate as a result of the genetic code with respect to a sequence defined in (a) or (b); or
(d) a sequence that is complementary to a polynucleotide defined in (a), (b) or (c);
   for use in a method of treatment of the human or animal body by therapy.

Typically a polynucleotide of the invention comprises a contiguous sequence of nucleotides which is capable of hybridizing under selective conditions to the complement of the coding sequence of SEQ ID NO: 2. Preferably a polynucleotide of the invention encodes a polypeptide which is capable of binding to a presenilin or retains a function of catenin p120.

A polynucleotide comprising a sequence that hybridizes to the complement of the coding sequence of SEQ ID NO: 2 can hydridize at a level significantly above background. Background hybridization may occur, for example, because of other cDNAs present in a cDNA library. The signal level generated by the interaction between a polynucleotide of the invention and the complement of the coding sequence of SEQ ID NO: 2 is typically at least 10 fold, preferably at least 100 fold, as intense as interactions between other polynucleotides and the coding sequence of SEQ ID NO: 2. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C.). However, such hybridization may be carried out under any suitable conditions known in the art (see Sambrook et al., 1989). For example, if high stringency is required, suitable conditions include 0.2×SSC at 60° C. If lower stringency is required, suitable conditions include 2×SSC at 60° C.

A nucleotide sequence which is capable of selectively hybridizing to the complement of the DNA coding sequence of SEQ ID NO: 2 will generally have at least 60%, at least 70%, at least 80%, at least 90%, at least 95%, at least 98% or at least 99% sequence identity to the coding sequence of SEQ ID NO: 2 over a region of at least 20, preferably at least 30, for instance at least 40, at least 60, more preferably at least 100 contiguous nucleotides or most preferably over the full length of SEQ ID NO: 2. Methods of measuring nucleic acid and protein homology are well known in the art.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al. 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10 μM=5, N=4, and a comparison of both strands.

The BLAST algorithm performs a statistical analysis of the similarity between two sequences; see e.g., Karlin and Altschul (1993) Proc. Natl. Acad. Sci. USA90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides for use in the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The coding sequence of SEQ ID NO: 2 may be modified by nucleotide substitutions, for example from 1, 2 or 3 to 10, 25, 50 or 100 substitutions. The polynucleotide of SEQ ID NO: 2 may alternatively or additionally be modified by one or more insertions and/or deletions and/or by an extension at either or both ends. The modified polynucleotide generally encodes a protein that can bind a presenilin. Degenerate substitutions may be made and/or substitutions may be made which would result in a conservative amino acid substitution when the modified sequence is translated, for example as shown in the Table above.

Polynucleotides of the invention may comprise DNA or RNA. They may also be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention.

Polynucleotides according to the invention may be produced recombinantly, synthetically, or by any means available to those of skill in the art. They may also be cloned by standard techniques. The polynucleotides are typically provided in isolated and/or purified form.

Although in general the techniques mentioned herein are well known in the art, reference may be made in particular to Sambrook et al, 1989, Molecular Cloning: a laboratory manual.

A polynucleotide may be an essential component in an assay of the invention, a probe (or template for designing a probe) for identifying proteins that may be used in the invention or a test compound. The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Antisense sequences, may also be used in gene therapy, such as in strategies for down regulation of expression of the proteins of the invention. The invention further provides double stranded polynucleotides comprising a polynucleotide for use in the invention and its complement for use in a method of treatment of the human or animal body by therapy.

Probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length.

Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 2.

Polynucleotides of the invention or for use in the invention can be inserted into expression vectors. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

Vectors for use in the invention may be transformed into a suitable host cell as described above to provide for expression of a polypeptide for use in the invention. Thus, in a further aspect the invention provides a process for preparing a polypeptide according to the invention which comprises cultivating a host cell transformed or transfected with an expression vector encoding the polypeptide, and recovering the expressed polypeptide.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of RNA or used to transfect or transform a host cell, for example, a mammalian host cell. The vectors may also be adapted to be used in vivo, for example in a method of gene therapy.

Promoters and other expression regulation signals may be selected to be compatible ith the host cell for which expression is designed. For example, yeast promoters include S. cerevisiae GAL4 and ADH promoters, S. pombe nmtl and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular endothelial or neuronal cell specific promoters (for example the DDAHI and DDAHII promoters), are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses (such as HPV-16 or HPV-18). Gene transfer techniques using these viruses are known to those skilled in the art. Retrovirus vectors for example may be used to stably integrate the polynucleotide giving rise to the antisense RNA into the host genome. Replication-defective adenovirus vectors by contrast remain episomal and therefore allow transient expression.

The invention also utilizes cells that have been modified to express a presenilin polypeptide and/or a catenin p120 polypeptide. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation of a polypeptide. A polypeptide of the invention may be overexpressed in bacterial cells, such as E. coli, and isolated from the bacterial culture.

According to another aspect, the present invention may also use antibodies (either polyclonal or preferably monoclonal antibodies, chimeric, single chain, Fab fragments) which have been raised by standard techniques and are specific for a presenilin polypeptide or for a catenin p120 polypeptide. Such antibodies could for example, be useful in purification, isolation or screening methods involving immunoprecipitation techniques and may be used as tools to further elucidate the function of catenin p120 or a variant thereof, or indeed as therapeutic agents in their own right. Antibodies may also be raised against specific epitopes of the proteins according to the invention. Such antibodies may be used to block ligand binding to the receptor. An antibody, or other compounds, “specifically binds” to a protein when it binds with preferential or high affinity to the protein for which it is specific but does not bind or binds with only low affinity to other proteins. A variety of protocols for competitive binding or immunoradiometric assays to determine the specific binding capability of an antibody are well known in the art (see for example Maddox et al 1993). Such immunoassays typically involve the formation of complexes between the “specific protein” and its antibody and the measurement of complex formation.

Antibodies may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example, an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Assays

Any suitable assay format may be used for identifying a modulator of a presenilin, for example a modulator of a presenilin/catenin p120 interaction. In a preferred aspect, the assay is a cell-based assay. Preferably the assay may be carried out in a single well of a microtitre plate. Assay formats which allow high throughput screening are preferred.

As the first step of the method for identifying a modulator of presenilin function, (a) a polypeptide comprising the sequence of SEQ ID NO: 1 or a variant or fragment of either sequence capable of binding to a presenilin; (b) a presenilin or a variant or a fragment thereof capable of binding to catenin p120; and (c) a test substance are contacted under conditions that would permit binding of (a) to (b) in the absence of a test substance. The activity of presenilin is then monitored. For example, the interaction between the polypeptide (a) and a presenilin (b) may be analysed. The interaction between the polypeptide (a) and presenilin (b) in the presence of a test substance may be compared with the interaction between the polypeptide (a) and presenilin (b) in the absence of the test substance to determine whether the test substance modulates the binding of polypeptide (a) and presenilin (b) and thereby whether the test substance enhances or inhibits the binding of a presenilin to catenin p 120.

As used herein, a polypeptide (a) is used to refer to catenin p120 having the sequence of SEQ ID NO: 1 or an isoform or variant thereof or a fragment of any thereof which is capable of binding to a presenilin, or to a variant or a fragment of presenilin which is capable of binding to catenin p120.

The test substance can be contacted with a cell harbouring a polynucleotide or expression vector encoding a polypeptide (a) and a polynucleotide or expression vector encoding a presenilin (b). Optionally the cell may harbour a polynucleotide or expression vector encoding a test substance, wherein the test substance is a peptide. The cell typically allows transcription and translation of the polynucleotides or vectors so that the polypeptides are expressed in the same cell.

The test substance may be provided in the extracellular medium used for washing, incubating or growing the cell. The test substance may modulate the interaction of presenilin (b) with the polypeptide (a) indirectly from outside the cell, for example by interacting with an extracellular domain of presenilin or may be taken up into the cell from the extracellular medium. Where presenilin (b) and polypeptide (a) are coexpressed in a cell, the cell may express both proteins naturally, for example the cell may be a neuronal cell grown in a primary culture, or the cell may express both proteins recombinantly, or the cell may naturally express one protein and be transformed to express the other protein recombinantly.

The cell may be transiently or stably transfected or transformed. The polypeptide (a), presenilin (b) and the test substance (c) where it is a peptide may all be stably expressed. More preferably polypeptide (a) and presenilin (b) will be stably expressed and a test substance peptide will be transiently expressed. Where only polypeptide (a) and presenilin (b) are expressed by the cell they may both be transiently expressed, both stably expressed or one may be stably expressed and the other transiently expressed. Cells can be transfected by methods well known in the art, for example, by electroporation, calcium phosphate precipitation, lipofection or heat shock. The proteins may be expressed in mammalian cells such as human cells or non-mammalian cells such as yeast or bacteria. It is preferred that the cells are in culture. Preferred cell lines which may be used include HEK293, COS and PC12 cells.

A polypeptide (a) and a presenilin (b) can also be recombinantly expressed in different cells. These cells may be two different cultures of the same cell line or may be different cell lines. The cell lines may both be mammalian cells, bacterial cells or yeast cells or the two cell lines may be from different organisms, for example a polypeptide (a) may be expressed in a mammalian cell and a presenilin (b) in a bacterial cell. A cell expressing a polypeptide (a) or a cell lysate, a membrane preparation or a protein preparation derived from a cell expressing a polypeptide (a) can be contacted with a cell expressing a presenilin (b) or a cell homogenate, a cell lysate or a protein preparation from cells expressing presenilin (b). Similarly, where the test substance is a peptide or protein, an expression vector comprising a polypeptide encoding (c) can be singly transfected into a cell and the cell homogenate, cell lysate, membrane preparation or protein preparation from the transfected cell can be used in the assay.

The conditions which permit binding of a polypeptide (a) to a presenilin (b) in an extracellular environment can be determined by carrying out the assay in the absence of a test substance.

A number of biochemical and molecular cell biology protocols known in the art can be used to analyse the interaction of a polypeptide (a) and presenilin (b) (see for example Sambrook et al., 1989). Some specific examples are outlined below:

The presenilin/catenin p120 interaction can be determined directly by incubating a radiolabelled polypeptide (a) with the presenilin and monitoring binding of the polypeptide (a) to the presenilin. Alternatively binding of radiolabelled presenilin to catenin p120 polypeptide (a) may be monitored. Typically, the radiolabelled substance can be incubated with cell membranes containing the presenilin or catenin p120 until equilibrium is reached. The membranes can then be separated from a non-bound substance and dissolved in scintillation fluid to allow the radioactive content to be determined by scintillation counting. Non-specific binding of the substance may also be determined by repeating the experiment in the presence of a saturating concentration of a non-radioactive polypeptide (a) or presenilin. Preferably a binding curve is constructed by repeating the experiment with various concentrations of the radiolabelled substance.

A yeast-2 hybrid assay system may be used. A polynucleotide encoding a presenilin, or fragment thereof capable of binding to catenin p120 can be cloned into GAL4 binding domain vector (GAL4_{BD}) and a polynucleotide comprising the sequence of SEQ ID NO: 2 or a variant or fragment thereof capable of binding to a presenilin can be cloned into a GAL4 activation domain fusion vector (GAL4_{AD}). Alternatively, a polynucleotide comprising the sequence of SEQ ID NO: 2 or a variant or fragment thereof capable of binding to a presenilin can be cloned into GAL4_{BD} and a polynucleotide encoding a presenilin can be cloned into GAL4_{AD}. GAL4_{AD} and GAL4_{BD} can then be expressed in yeast and the resulting β-galactosidase activity can be assayed and quantified using the substrate o-nitrophenol β-D-galactopyranoside (ONPG) using a liquid nitrogen freeze fracture regime as described by Harshman et al., 1998.

A “pull-down” assay system may also be used. A presenilin, or a variant or fragment thereof capable of binding a catenin p 120 can be run on a denaturing SDS polyacrylamide gel and transferred to a nitrocellulose membrane. Use of a fusion protein, such as a GST-fusion protein expressed and purified from E. coli, is preferred. The protein on the gel can then be renatured. A number of protocols for the refolding of denatured proteins are detailed in Marston (1987). In a parallel experiment, the position of the presenilin can be identified by immunoblotting using techniques well known in the art. A labelled polypeptide (a) capable of interacting with a presenilin, which may be present, for example, in a cell extract from cells transiently transfected with a polynucleotide of SEQ ID NO: 2 capable of interacting with a presenilin grown in medium containing ³⁵S-methionine, can then be incubated with the nitrocellulose membrane. The test compound can be included in the incubation medium. After washing to remove non-specifically bound proteins, labelled catenin p120 bound to the presenilin can be detected and quantified using, for example, a phosphorimager or a scintillation counter. The assay can also be carried out by immobilizing the catenin p120 and measuring the binding of a presenilin to the immobilized protein.

Alternatively, a presenilin or variant or fragment thereof capable of binding catenin p120, a polypeptide comprising SEQ ID NO: 1 or a variant or fragment thereof capable of interacting with a presenilin may be immunoprecipitated, immunopurified or affinity purified from a cell extract of cells co-expressing a presenilin or fragment thereof and a polypeptide comprising SEQ ID NO: 1 or a variant or fragment of either sequence capable of binding to a presenilin. If the test substance is a polypeptide the cells may also co-express the test substance. Alternatively, the test substance may be provided in the cell growth medium. Coprecipitating/copurifying catenin p120 or presenilins can then be detected, for example using Western blotting techniques or by radiolabelling recombinantly expressed proteins, and quantified using a phosphorimager or scintillation counter.

An important aspect of the present invention is the use of catenin p120 polypeptides according to the invention in screening methods to identify compounds that may act as modulators of catenin p120 activity and in particular compounds that may be useful in treating presenilin associated disease. Any suitable form may be used for the assay to identify a modulator of catenin p120 activity. In general terms, such screening methods may involve contacting a polypeptide of the invention with a test compound and then measuring activity.

Any suitable activity of catenin p120 may be measured in a screening assay. For example, the binding of catenin p120 to the cystoskeleton or to a transcription factor may be monitored. A modulator may, for example, modulate binding of catenin p120 to E-cadherin and/or may modulate binding of catenin p120 to a transcription factor such as Kaiso (Daniel and Reynolds, 1999). Alternatively, catenin p120 activity may be monitored by detecting expression of a reporter gene, such as luciferase, under the control of a regulatory sequence which binds a catenin p120/transcription factor protein complex or a transcription factor which is activated by catenin p120.

A ligand of catenin p120 can be determined directly by incubating a radiolabelled test substance with the polypeptide (a) and monitoring binding of the test compound to the polypeptide. Typically, the radiolabelled test substance can be incubated with cell membranes containing the polypeptide until equilibrium is reached. The membranes can then be separated from a non-bound test substande and dissolved in scintillation fluid to allow the radioactive content to be determined by scintillation counting. Non-specific binding of the test substance may also be determined by repeating the experiment in the presence of a saturating concentration of a non-radioactive ligand. Preferably a binding curve is constructed by repeating the experiment with various concentrations of the test substance.

Modulator activity can be determined by contacting cells expressing a polypeptide (a) of the invention with a substance under investigation and by monitoring the effect mediated by the polypeptide. The cells expressing the polypeptide may be in vitro, for example in cultured cells, or in vivo. The polypeptide of the invention may be naturally or recombinantly expressed. Preferably, the assay is carried out in vitro using cells expressing recombinant polypeptide.

For each assay system a parallel control experiment (in which the substance to be tested is omitted) and experiments in which a test substance is included can be carried out. The results of the experiments using the test compound and the control experiments can be used to determine whether the test compound inhibits or enhances binding.

The substance tested may be tested with any other known receptor/interacting cytoplasmic protein combinations, for example presenilin and β-catenin, to exclude the possibility that the test substance is a general inhibitor of protein/protein interactions.

Where a variant or fragment of presenilin or catenin p120 is used in the assay as the presenilin (b) or polypeptide (a), the assay is preferably run first in the absence of a test substance to ensure that the variant or fragment does not affect the activity of the presenilin or catenin p120. The assays may also be carried out monitoring PS mediated signalling. For example, the step of monitoring presenilin activity may involve assessment of presenilin mediated signalling or processing or the effect of binding of presenilin to other proteins. For example the assay may involve determination of APP processing, Notch signalling, for example in early is development, or the binding of presenilin to tau and a tau kinase.

Candidate Modulators

A modulator of presenilin or catenin p 120 function may exert its effect by binding directly to presenilin or catenin p120 polypeptide or may have an upstream effect which prevents the presenilin/catenin p120 interaction occurring, or presenilin or catenin p120 mediated activity.

A modulator may directly inhibit the interaction of presenilin with catenin p120 or inhibit interaction between catenin p120 and a ligand such as E-cadherin. A candidate modulator may comprise a fragment of a catenin p120 isoform capable of binding a presenilin or catenin p120 ligand but lacking any functional activity. Alternatively, a candidate modulator may comprise a fragment of a presenilin capable of binding catenin p120 but lacking any functional activity. Candidate molecules include N-terminal and C-terminal fragments of presenilin 1 or presenilin 2. Antibodies or antibody fragments, for example as defined herein, that specifically bind to presenilin or catenin p120 or chemical compounds capable of binding these proteins are also candidate compounds.

Other suitable test substances include combinatorial libraries, defined chemical entities and compounds, peptide and peptide mimetics, oligonucleotides and natural product libraries, such as display libraries (e.g. phase display libraries).

Typically, organic molecules will be screened, preferably small organic molecules which have a molecular weight of from 50 to 2500 daltons. Candidate products can be biomolecules including, saccharides, fatty acids, steroids, purines, pyrimidines derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 10 mM, preferably from, 100 μM to 1000 μM or from 1 μM to 100 μM, more preferably from 1 μM to 10 μM.

Modulators

A modulator of presenilin activity which produces a measurable reduction or increase in catenin p120 to presenilin in the assays described above, or an effect on presenilin activity or catenin p120 mediated activity.

Preferred inhibitors are those which inhibit binding by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 g ml⁻¹, 1 mg ml⁻¹, 10 mg ml⁻¹ or 100 mg ml⁻¹.

Preferred activators are those which activate binding by at least 10%, at least 25%, at least 50%, at least 100%, at least, 200%, at least 500% or at least 1000% at a concentration of the activator 1 μg ml⁻¹, 10 μg ml⁻¹, 100 μg ml⁻¹, 500 μg ml⁻¹, 1 mg ml⁻¹, 10 mg ml⁻¹ or 100 mg ml⁻¹.

The percentage inhibition or activation represents the percentage decrease or increase in expression/activity in a comparison of assays in the presence and absence of the test substance. Any combination of the above mentioned degrees of percentage inhibition or activation and concentration of inhibitor or activator may be used to define an inhibitor or activator of the invention, with greater inhibition or activation at lower concentrations being preferred.

Candidate substances which show activity in assays such as those described above can be tested in in vivo systems, an animal model. Candidate inhibitors could be tested for their ability to decrease presenilin mediated signalling, for example by interfering with development in C. Elegans or by interfering with APP processing or by monitoring changes in gene expression using a reporter gene assay.

Candidate activators could be tested for their ability to increase presenilin mediated signalling. Ultimately such substances would be tested in animal models of the target disease states.

Therapeutic Use

Modulators of presenilin/catenin p120 or of presenilin activity or of catenin p120 activity identified by the methods of the invention may be used for the treatment or prophylaxis of a disorder that is responsive to modulation of presenilin activity or catenin p120.

In particular, neuronal disorders such as cognitive disorders including Alzheimer's disease may be treated. A modulator of presenilin or catenin p120 activity may be used to alleviate the symptoms or to improve the condition of a patient suffering from such a disorder. A therapeutically effective amount of a modulator is an amount which is sufficient to alleviate one or more symptoms of a disorder or to improve the condition of a patient suffering from a disorder.

Modulators of presenilin or catenin p120 activity may be useful in enhancing cognitive function. Hence, a therapeutically effective amount of a modulator may be an amount which is sufficient to produce an enhancement of cognitive function in a patient suffering from a neurodegenerative disorder. This may be useful in treating neurodegenerative diseases such as Alzheimer's disease or in enhancing cognitive function following injury to the brain.

Catenin p120 polypeptides and polynucleotides as described herein may also be used in the treatment or prophylaxis of such disorders.

Another aspect of the present invention is the use of polynucleotides encoding the catenin p120 polypeptides of the invention to identify mutations in catenin p120 genes which may be implicated in human disorders. Identification of such mutations may be used to assist in diagnosis of or susceptibility to Alzheimer's or other conditions associated with presenilin and in assessing the physiology of such disorders. Polynucleotides may also be used in hybridisation studies to monitor for expression of catenin p120 genes and in particular for up or down regulation of catenin p120 expression.

The present invention provides a method for assessing a disorder associated with abnormal presenilin function such as Alzheimer's disease by detecting variation in the expressed products encoded by a catenin p120 gene. This may comprise determining the level of an catenin p120 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction with kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo, G. J., 1992, “PCR In Situ Hybridization: Protocols And Applications”, Raven Press, NY). Such analysis techniques include, DNA or RNA blotting analyses, single stranded conformational polymorphism analyses, in situ hybridization assays, and polymerase chain reaction analyses. Such analyses may reveal both quantitative aspects of the expression pattern of catenin p120, and qualitative aspects of catenin p120 expression and/or composition.

Alternative diagnostic methods for the detection of catenin p120 nucleic acid molecules may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. 15 USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large numbers of nucleic acid sequence targets rapidly and accurately. By tagging with oligonucleotides or using fixed probe arrays, one can employ chip technology to segregate target molecules as high density arrays and screen these molecules on the basis of hybridization.

Following detection, the results seen in a given patient may be compared with a statistically significant reference group of normal patients and patients that have presenilin related pathologies. In this way, it is possible to correlate the amount or kind of catenin p120 polypeptide detected with various clinical states or predisposition to clinical states.

Substances identified according to the screening methods outlined above may be formulated with standard pharmaceutically acceptable carriers and/or excipients as is routine in the pharmaceutical art. For example, a suitable substance may be dissolved in physiological saline, water or an isotonic solution for injections. The exact nature of a formulation will depend upon several factors including the particular substance to be administered and the desired route of administration. The formulation will also depend on whether a pharmaceutical or veterinary use is intended. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical, parenteral, subcutaneous, intravascular, transdermal or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Modulators may have to be administered to specific sites, or otherwise targeted to brain cells. For example, the modulator may be delivered to neurons. This may be achieved, for example, by delivery via a viral strain such as herpes simplex virus. When the polynucleotide of the invention is delivered to cells by a viral vector, the amount of virus administered is in the mage of from 10⁶ to 10¹⁰ pfu, preferably from 10⁷ to 10⁹ pfu, more preferably about 10⁸ pfu for adenoviral vectors. When injected, typically 1-2 ml of virus in a pharmaceutically acceptable suitable carrier or diluent is administered. The vector may comprise a promoter or other regulatory sequence that is specific to certain neurons.

Nucleic acid encoding catenin p120 or presenilin or a variant or fragment thereof which inhibits the presenilin/catenin p120 interaction or other catenin p120 activity may be administered to the mammal. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably transdermally, intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Where the polynucleotide giving rise to the product is under the control of an inducible promoter, it may only be necessary to induce gene expression for the duration of the treatment. Once the condition has been treated, the inducer is removed and expression of the polypeptide of the invention ceases. This will clearly have clinical advantages. Such a system may, for example, involve administering the antibiotic tetracycline, to activate gene expression via its effect on the tet repressor/VP16 fusion protein.

The use of tissue-specific promoters will be of assistance in the treatment of disease using the polypeptides, polynucleotide and vectors of the invention. It will be advantageous to be able express therapeutic genes in only the relevant affected cell types, especially where such genes are toxic when expressed in other cell types.

The routes of administration and dosages described above are intended only as a guide since a skilled physician will be able to determine readily the optimum route of administration and dosage for any particular patient and condition. The following Examples illustrate the invention.

### EXAMPLE 1

### Interaction of PS-1 and Catenin p120 in HEK293 Cells

The target protein presenilin 1 (Accession Number L42110, Hugo Number PSEN1, Unigene Number Hs.3260) was amplified by PCR using primers GGAAGTGGAAGTGGCACAGAGTTACCTGCACCGTTGTCC and GGAGGTTGGATTGGCTTAGATATAAAA TTGATGGAATGC to give a 1439 bp PCR product. The PCR product was cloned into an expression vector such that a single HA tag (sequence AYPYDVPDYA) was inserted at the N-terminus. This construct was transfected into HEK293 cells that were expanded under conditions selecting for the expression construct. In a representative experiment, presenilin was immunoprecipitated from 65 mg of membrane-enriched fraction derived from approximately 10⁸ transfected cells. Antibodies used were 80 μg mouse monoclonal antibody HA11 (recognising the HA tag) (BABCO) or 30 μg rat monoclonal antibody to presenilin 1 (Chemicon mAb 1563). Immunoprecipitates were recovered using 200 μl of a 50/50 slurry of protein G sepharose (Pharmacia), resuspended in sample buffer, separated on a 4-20% tris glycine gel under non-reduced conditions and stained with colloidal Coomassie blue. Bands specific to the tagged cell line were excised and in gel digested with trypsin. All trypsin digested peptides were subjected first to matrix assisted laser desorption ionization time of flight (MALDI-TOF) mass spectrometry and in a number of cases this was sufficient to make a positive identification by comparing peptide mass data with that predicted from non-redundant protein databases. In all cases peptides were further subjected to LC/MS/MS to further confirm the MALDI identification or to identify proteins not detected by MALDI-TOF, again using a non-redundant protein database. Catenin p120 was identified as interacting with presenilin 1.

Without complete sequence coverage by mass spectrometry it is not possible to inconclusively determine which p120 isoform is present. One analysis that demonstrates that the isoform present could be either 1AC or 1A (exon 20 absent, exon 18 present, presence/absence of exon 11 not established).

### EXAMPLE 2

### Interaction of PS01 and KIAA0253 in PC12 Cells, a Neuronal Cell Line

The HA tagged human presenilin 1 construct was transfected into PC12 cells. Stable transfectants were generated and expanded under conditions selecting for the expression construct. In a representative experiment presenilin-1 was affinity-purified from 75 mg of membrane-enriched fraction derived from approximately 10⁸ cells. Membrane extracts were prepared by homogenising cell pellets in a blender, taking the supernatant from a low-speed (1200 rpm/10 min) spin and subjecting it to a 100,000 g/60 min spin. The resulting membrane-enriched pellet was resuspended in extraction buffer and pass through a 26 g needle. The solution then made 1% with dodecyl maltoside and mixed for 1 hour at 4° C. The solution was then re-spun at 100,000 g for 30 mins to clear precipitating debris. The antibody used was mouse monoclonal antibody HA11 (recognising the HA tag) (BABCO) covalently linked to sepharose beads (Perbio aminolink). Membrane extract was incubated with sepharose-bound antibody overnight at 4° C. Beads were then washed 6 times with extraction buffer plus 0.4 mM dodecyl maltoside. Proteins that remained bound to the sepharose-antibody beads were then eluted with 100 μl sample buffer (Novex) and run on a pre-cast 4-12% ID SDS PAGE gel in MOPS buffer (Novex) under reducing conditions and stained with colloidal Coomassie blue. Bands specific to the tagged cell line were excised and in-gel digested with trypsin. Trypsin-digested peptides were subjected to LC/MS/MS for protein identification, using a non-redundant protein database. Catenin p120 was identified as interacting with presenilin 1 in PC 12 cells.

This result extends the interaction of PS-1 and catenin p120 to a neuronal cell type that might be considered more relevant to processes involved in neurodegenerative disorders such as Alzheimer's disease.

### REFERENCES

Anderton B. H. 1999. Curr. Biol. 9: 106-9.
Capell A. Grunberg J. Pesold B. Diehlmann A. Citron M. Nixon R. Beyreuther K.
Selkoe D J. Haass C. 1998 J. Biol. Chem. 273: 3205-11.
Daniel J. M. and Reynolds A. B. 1999. Mol. Cell. Biol. 19(5): 3614-23.
Kang D. E., Soriano, S., Frosch M. P., Collins, T., Naruse, S., Sisodia, S S., Leibowitz,
G., Levine, F., Koo, E. H. 1999. J. Neurosci. 19: 4229-37
Keirsebilck, A., Bonneacute, S., Staes, K., Hengel, J., Nollet, F., Reynolds, A., van Roy, F. 1998. Genomics 50: 129-146
Haass, C. and Mandelkow, E. 1999. Trends in Cell Biol. 9: 241-244
Levesque G., Yu, G., Nishimura, M., Zhang, D. M., Levesque, L., Yu, H., Xu, D., Liang, Y., Rogaeva, E., Ikeda, M., Duthie, M., Murgolo, N., Wang, L., VanderVere, P., Bayne, M. L., Strader, C. D., Rommens, J. M., Fraser, P. E., St. George-Hyslop, P. 1999 J. Neurochem. 72: 999-1008
Ray, W. J., Yao, M., Nowotny, P., Mumm., J., Zhang, W., Wu, J., Y., Kopan, R., Goate, A. M. 1999. Proc. Natl. Acad. Sci. USA 96: 3263-8
Selkoe, D. J. 1998. Trends in Cell Biol. 8: 447-53
Stahl, B., Diehlmann, A., Sudhof, T. C. 1999 J. Biol. Chem. 274: 9141-9148
Takashima, A., Murayama, M., Murayama, O., Kohno, T., Honda, T., Yasutake, K., Nihonmatsu, N., Mercken, M., Yamaguchi, H., Sugihara, S., Wolozin, B. 1998. Proc. Natl. Acad. Sci. USA 9: 9637-41
Weidemann, A., Paliga, K., Durrwang, U., Czech, C., Evin, G., Masters, C. L., Beyreuther, K. 1997. Nat. Med. 3: 328-32
Xia, W., Zhang, J., Perez, R., Koo, E. H., Selkoe, D. J. 1997. Proc. Natl. Acad. Sci. USA 94: 8208-13
Yu, G., Chen, F., Levesque, G., Nishimura, M., Zhang, D. M., Levesque, L., Rogaeva, E., Xu, D., Liang, Y., Duthie, M., St George-Hyslop, P. H., Fraser P. E. 1998. J. Biol. Chem. 273: 16470-5
Zhang, Z., Hartmann, H., Do, V. M., Abramowski, D., Sturchler-Pierrat, C., Staufenbiel, M., Sommer, B., van de Wetering, M., Clevers, H., Saftig, P., De Strooper, B., He, X., Yankner, B. A. 1998. Nature. 395: 698-702
Zhang, W., Han, S. W., McKeel, D. W., Goate, A., Wu, J. Y. 1998 J. Neurosci. 18: 914-22
Zhou, J., Liyanage, U., Medina, M., Ho, C., Simmons, A. D., Lovett, M., Kosik, K. S. 1997a. Neuroreport. 8: 2085-90
Zhou J. Liyanage U. Medina M. Ho C. Simmons A D. Lovett M. Kosik K S. 1997b. Neuroreport. 8: 1489-94

1. A method of identifying a modulator of presenilin function, the method comprising:
(i) providing
(a) a polypeptide capable of binding presenilin, which polypeptide comprises the amino acid sequence of SEQ ID NO: 1 or a fragment thereof which is capable of binding to presenilin;
(b) a presenilin or a fragment thereof capable of binding to a polypeptide which comprises the amino acid sequence of SEQ ID NO: 1;
(c) a test substance
under conditions that would permit binding of a polypeptide (a) to a presenilin (b) in the absence of the test substance;
(ii) monitoring presenilin mediated activity; and
(iii) determining thereby whether the test substance is a modulator of presenilin activity.

2. A method according to claim 1 wherein step (ii) comprises monitoring the interaction between polypeptide (a) and presenilin (b).

3. A method according to claim 2 wherein the modulator inhibits the binding of the polypeptide (a) to the presenilin (b).

4. A method according to claim 2 wherein the modulator enhances the binding of the polypeptide (a) to the presenilin (b).

5. A method according to claim 1 wherein the presenilin (b) is presenilin 1 or a said fragment thereof.

6. A method according to claim 1 wherein step (i) comprises:
(a) transfecting a cell with polynucleotides encoding a polypeptide (a) and a presenilin (b);
(b) allowing the said cell to express the polypeptide (a) and presenilin (b); and
(c) contacting the said cell with a test substance.

7. A method according to claim 1 wherein step (i) comprises:
(a) transfecting a cell with polynucleotides encoding a polypeptide (a), a presenilin (b) and a test substance (c) which is a peptide and
(b) allowing the said cell to express the polypeptide (a), presenilin (b) and the peptide test substance (c).

8. A method according to claim 1 wherein step (i) comprises:
(a) transfecting a first cell with a polynucleotide encoding a polypeptide (a);
(b) transfecting a second cell with a polynucleotide encoding a presenilin (b);
(c) allowing said first cell to express the polypeptide (a) and said second cell to express the presenilin (b);
(d) preparing a cell extract from each of said first and second cell; and
(e) contacting said cell extract from first cell and said cell extract from second cell in the presence of a test substance.

9. A method for identification of a compound that modulates catenin p120 activity, which method comprises:
(i) contacting a catenin p120 polypeptide comprising
(a) the amino acid sequence of SEQ ID NO: 1; or
(b) a fragment thereof which maintains a catenin p120 function; with a test compound and
(ii) monitoring for catenin p120 activity thereby determining whether the test compound is a modulator of catenin p120.

10. A method according to claim 9 wherein the catenin p120 activity comprises the ability of the polypeptide to interact with a presenilin, or a fragment thereof.

11. (canceled)

12. (canceled)

13. (canceled)

14. (canceled)

15. (canceled)

16. (canceled)
